(19) United States
(12) Patent Application Publication
(10) Pub. No.: US 2024/0164825 A1
(43) Pub. Date: May 23, 2024

(54) EXOTHERMIC REACTION-BASED ABLATION

(71) Applicant: Medtronic Ireland Manufacturing Unlimited Company, Dublin 2 (IE)

(72) Inventors: Manoj Kumar Singh, Santa Rosa, CA (US); Stefan S. Tunev, Santa Rosa, CA (US)

(21) Appl. No.: 18/552,750

(22) PCT Filed: Mar. 31, 2022
(86) PCT No.: PCT/EP2022/058684

§ 371 Date: Sep. 27, 2023

Related U.S. Application Data
(60) Provisional application No. 63/168,872, filed on Mar. 31, 2021.

(51) Int. Cl. A61B 18/06; A61B 17/00; A61B 18/00; A61K 33/06; A61L 29/10; A61L 29/16; A61M 25/10

(52) U.S. Cl. CPC A61B 18/06; A61K 33/06; A61L 29/106; A61L 29/16; A61B 2017/00004; A61B 2018/00154; A61B 2018/0022; A61B 2018/00267; A61B 2018/00404; A61B 2018/00434; A61B 2018/00511; A61B 2018/00577; A61B 2018/068; A61L 2400/12; A61M 2025/105

(57) ABSTRACT

Various embodiments of ablation elements are disclosed herein, where the ablation element includes a biodegradable shell. Calcium chloride is retained within the biodegradable shell. When the ablation element is delivered to a target location within the body of a patent, the biodegradable shell degrades to release the calcium chloride. A reaction between the released calcium chloride and bodily fluids will generate heat, which in turn will ablate biological material in proximity to the reactive region.

### FIELD

The present disclosure generally relates to the field of ablation of biological material of a patient and, more particularly, to an exothermic reaction-based modality for ablation.

### BACKGROUND

The nerves that lead to a patient's kidneys are part of the patient's sympathetic nervous system. An overactive sympathetic nervous system has been identified as a mechanism that results in high blood pressure. Various modalities have been used to interrupt the signaling associated with a patient's renal nerves, and this therapy may be referred to as “denervation.” Representative renal denervation modalities include RF energy, pulsed electrical energy, microwave energy, optical energy, ultrasound energy (e.g., intravascularly delivered ultrasound, and/or HIFU), direct heat energy, radiation (e.g., infrared, visible, and/or gamma radiation), cryo-therapeutic cooling, and chemical ablation.

### SUMMARY

A number of what may be characterized as ablation elements are presented herein. Each such ablation element utilizes/incorporates a biodegradable shell with an ablation agent enclosed within the biodegradable shell. Degradation of the biodegradable shell, when at a target location within a human body, releases the ablation agent. A reaction between the released ablation agent and one or more bodily fluids may generate heat that may be used to ablate at least one or more nerves, to ablate a tumor, or to ablate tissue, in at least certain instances the reaction may be in the form of a chemical reaction that results in necrosis (typically for extravascular applications), or both. A preferred ablation agent is calcium chloride, although one more or appropriate agents/materials may be enclosed within the biodegradable shell to provide the above-noted heating and/or necrosing features upon release. Both the configuration of such an ablation element and the use of such an ablation element are within the scope of this Summary.

One aspect of an ablation element in accordance with the foregoing has the biodegradable shell including first and second end walls and an annular sidewall, with the first and second end walls being spaced along a length dimension of the biodegradable shell, and with the annular sidewall extending between the first and second end walls. The noted ablation agent is enclosed within the biodegradable shell defined by the first and second end walls and the annular sidewall. One embodiment has at least a portion of a length of the annular sidewall being of a reduced wall thickness compared to the first and second end walls, such that the ablation agent will be at least initially released along this reduced wall thickness portion of the annular sidewall. Another embodiment has the annular sidewall including at least one groove, such that the ablation agent will be at least initially released at a location corresponding with this at least one groove. A plurality of annular grooves may be disposed along the length dimension of the biodegradable shell. A plurality of axially-extending grooves may extends along the length dimension of the biodegradable shell. A groove may extend along the length dimension of the biodegradable shell, for instance in a helical or spiral fashion. Another embodiment has the first and second end walls being formed from a different biodegradable material(s) compared to the annular sidewall, with the biodegradable material(s) forming the annular sidewall being degradable at a higher/faster rate than a biodegradable material(s) that forms the first end wall and the second end wall. The annular sidewall of the biodegradable shell could be cylindrical or could taper proceeding from the first end wall to the second end wall. A press fit may exist between at least part of the annular sidewall of the biodegradable shell and a wall of a vessel of a patient when the ablation element is delivered to a target location.

One aspect of an ablation element in accordance with the foregoing has the biodegradable shell including first and second ends, an annular outer sidewall, and an annular inner sidewall with the first and second ends being spaced along a length dimension of the biodegradable shell, with the annular inner and outer sidewalls each extending between the first and second ends, and with an opening extending entirely through the biodegradable shell proceeding along its length dimension (e.g., the opening extending from the first end of the biodegradable shell to the second end of the biodegradable shell; the annular inner sidewall of the biodegradable shell defining a perimeter of the opening). The noted ablation agent is enclosed within the biodegradable shell by the first and second ends, the annular inner sidewall, and the annular outer sidewall. One embodiment has the annular inner sidewall and the annular outer sidewall being formed from different biodegradable materials, with the biodegradable material(s) forming the annular outer sidewall being degradable at a higher/faster rate than the biodegradable material(s) forming the annular inner sidewall. The annular outer sidewall of the biodegradable shell could be cylindrical or could taper proceeding from the first end to the second end. A press fit may exist between at least part of the annular outer sidewall of the biodegradable shell and a wall of a vessel of a patient when the ablation element is delivered to a target location. The opening through the ablation element accommodates a flow of bodily fluids through the ablation element when the ablation element has been delivered to a target location within a vessel of a patient.

One aspect of an ablation element in accordance with the foregoing has the biodegradable shell being of a spherical configuration. Although the ablation element in this case could be disposed within a vessel of a patient, this ablation element may be disposed at one or more target locations that are external to a vessel of a patient (e.g., extravascularly disposed at a target location and any appropriate ablation agent, including an ablation agent that provides a chemical ablation). Such a spherical biodegradable shell may be an outer diameter of 1 mm or less.

One aspect of an ablation element in accordance with the foregoing entails at least part of a catheter shaft incorporating such an ablation element. An entirety of a distal end section of the catheter shaft may be in the form of the ablation element. The biodegradable shell of an ablation element could also be disposed on an exterior of at least part of the catheter shaft (e.g., at one or more locations along the length of the catheter shaft). The catheter may be disposed in a deployed configuration after being delivered to a target location to dispose the biodegradable shell in contact with a wall of a vessel of a patient.

One aspect of an ablation element in accordance with the foregoing includes incorporating such an ablation element on at least part of an exterior or outer perimeter of an expandable element, such as a stent. The stent may include what may be characterized as a skeleton or skeletal framework, and one or more openings may be distributed throughout this skeleton. One option is for the biodegradable shell to be incorporated only on one or more portions of an exterior of the skeleton for the stent. Another option is for the biodegradable shell to be disposed about at least one annular portion of the stent (e.g., over the corresponding portion of the skeleton and any corresponding openings; the biodegradable shell could be disposed about the entirety of the stent). The stent may be disposed in a deployed configuration after being delivered to a target location to dispose the biodegradable shell in contact with a wall of a vessel of a patient.

One aspect of an ablation element in accordance with the foregoing includes disposing the biodegradable shell about an annular outer sidewall of an expandable element, such as a balloon. The calcium chloride may be retained within a space between the balloon and the biodegradable shell.

An ablation system that does not use an ablation element in accordance with the foregoing, instead utilizes an expandable balloon. A first feed lumen, a second feed lumen, and an exhaust lumen each extend into an interior of this balloon. A first feed source is connected with the first lumen, and includes an ablation agent (e.g., calcium chloride). A second feed source is connected with the second lumen and includes a liquid. The balloon may be delivered to a target location within a vessel of a patient (e.g., in a delivery configuration). The ablation agent and the liquid may then be directed into the balloon to inflate the same into contact with a wall of the vessel to dispose the balloon into contact with the wall of the vessel. Heat generated by a reaction of the ablation agent with the liquid will heat the balloon, which in turn may be used to ablate a region surrounding the vessel (e.g., to ablate one or nerves in this region).

Various aspects of the present disclosure are also addressed by the following paragraphs and in the noted combinations:
1. A method of ablating a biological material, comprising:
delivering an ablation element into a body of a patient, wherein said ablation element comprises a biodegradable shell and calcium chloride retained within said biodegradable shell;
releasing said calcium chloride from said biodegradable shell after said delivering; and
ablating biological material in proximity to said biodegradable shell after said releasing.
2. The method of paragraph 1, wherein said delivering is selected from the group consisting of an intravascular delivery and an extravascular delivery.
3. The method of paragraph 1, wherein said biodegradable shell comprises first and second end walls and an annular sidewall, wherein said first and second end walls are spaced along a length dimension of said biodegradable shell and with said annular sidewall extending between said first and second end walls.
4. The method of paragraph 3, wherein said delivering comprises disposing said annular sidewall in interfacing relation with a wall of a vessel of said patient.
5. The method of any of paragraphs 3-4, wherein at least a portion of a length of said annular sidewall has a smaller wall thickness than each of said first and second end walls.
6. The method of paragraph 5, wherein said releasing comprises degrading at least a portion of said annular sidewall of said biodegradable shell prior to degrading said first and second end walls of said biodegradable shell.
7. The method of any of paragraphs 3-4, wherein said annular sidewall comprises at least one groove.
8. The method of paragraph 7, wherein said at least one groove is disposed on an exterior of said annular sidewall.
9. The method of any of paragraphs 7-8, wherein said at least one groove is selected from the group consisting of: a plurality of annular grooves that are spaced along said length dimension of said biodegradable shell; at least one groove that spirals proceeding along said length dimension of said biodegradable shell; a plurality of axially extending grooves; and any combination thereof.
10. The method of any of paragraphs 7-8, wherein said plurality of axially-extending grooves are each at least substantially parallel with a central axis of said ablation element.
11. The method of any of paragraphs 7-10, wherein said releasing comprises degrading said biodegradable shell at a location corresponding with said at least one groove prior to degrading a remainder of said biodegradable shell.
12. The method of any of paragraphs 3-11, further comprising providing a press fit between said biodegradable shell and said wall of said vessel.
13. The method of any of paragraphs 3-12, wherein a form of said calcium chloride is selected from the group consisting of powder, pallets, and beads.
14. The method of any of paragraphs 1-2, wherein said biodegradable shell is spherical.
15. The method of paragraph 14, wherein said biodegradable shell is disposed in patient tissue after said delivering.
16. The method of any of paragraphs 14-15, wherein a form of said calcium chloride is selected from the group consisting of powder, pallets, beads, and a liquid.
17. The method of paragraph 1, wherein said biodegradable shell comprises first and second ends that are spaced along a length dimension of said biodegradable shell, an annular outer sidewall that extends between said first and second ends, an annular inner sidewall that is spaced inwardly of said annular outer sidewall and that extends between said first and second ends, and an opening that extends entirely through said biodegradable shell proceeding along said length dimension, wherein said opening intersects and proceeds through each of said first and second ends, and wherein an outer perimeter of said opening is defined by said annular inner sidewall.
18. The method of paragraph 17, wherein said delivering comprises disposing said annular outer sidewall in interfacing relation with a wall of a vessel of said patient.
19. The method of paragraph 18, further comprising providing a press fit between said biodegradable shell and said wall of said vessel.
20. The method of any of paragraphs 17-19, wherein said annular outer sidewall is formed from a first biodegradable material, said annular inner sidewall is formed from a second biodegradable material, and said first biodegradable material degrades at a higher rate than said second biodegradable material.
21. The method of any of paragraphs 17-20, wherein a form of said calcium chloride is selected from the group consisting of powder, pallets, and beads.
22. The method of paragraph 1, wherein said ablation element is associated with a catheter shaft.
23. The method of paragraph 22, wherein a form of said calcium chloride is selected from the group consisting of powder, pallets, beads, and liquid.
24. The method of any of paragraphs 22-23, wherein said ablation element extends distally from a distal end of said catheter shaft.
25. The method of any of paragraphs 22-23, wherein said ablation element is disposed on an exterior of said catheter shaft.
26. The method of paragraph 1, further comprising:
expanding an expandable element within a vessel of said patient, wherein said biodegradable shell is disposed on at least a portion of an outer perimeter of said expandable element, and wherein said expanding comprises disposing said biodegradable shell in contact with a wall of said vessel.
27. The method of paragraph 26, wherein said expandable element is a stent.
28. The method of paragraph 27, wherein said stent comprises a skeleton and a plurality of openings throughout said skeleton.
29. The method of paragraph 28, wherein said biodegradable shell is incorporated only on an exterior of said skeleton.
30. The method of paragraph 28, wherein said biodegradable shell is disposed about an annular portion of said outer perimeter of said stent.
31. The method of paragraph 1, wherein said biodegradable shell is disposed about an annular outer sidewall of an expandable element, wherein said calcium chloride is enclosed in a space between said biodegradable shell and said annular outer sidewall of said expandable element, said method further comprising:
expanding said expandable element within a vessel of said patient to dispose said biodegradable shell in contact with a wall of said vessel.
32. The method of paragraph 31, wherein said expandable element is a balloon.
33. The method of any of paragraphs 1-32, wherein said releasing comprises degrading at least a portion of said biodegradable shell.
34. The method of any of paragraphs 1-33, wherein said ablating comprises at least one of ablating at least one nerve, ablating a tumor, and ablating tissue.
35. The method of any of paragraphs 1-34, wherein said ablating comprises reacting said calcium chloride with at least one bodily fluid after said releasing, said method further comprising generating heat from said reacting.
36. An ablation element for a biological material, comprising:
a biodegradable shell comprising first and second end walls and an annular sidewall, wherein said first and second end walls are spaced along a length dimension of said biodegradable shell and with said annular sidewall extending between said first and second end walls; and
calcium chloride retained within said biodegradable shell;
wherein at least a first length section of said annular sidewall has a smaller wall thickness than each of said first and second end walls.
37. An ablation element for a biological material, comprising:
a biodegradable shell comprising first and second ends and an annular sidewall, wherein said first and second ends are spaced along a length dimension of said biodegradable shell and with said annular sidewall extending between said first and second ends; and
calcium chloride retained within said biodegradable shell;
wherein said annular sidewall comprises at least one groove.
38. An ablation element for a biological material, comprising:
a biodegradable shell comprising first and second ends that are spaced along a length dimension of said biodegradable shell, an annular outer sidewall that extends between said first and second ends, an annular inner sidewall that is spaced inwardly of said annular outer sidewall and that extends between said first and second ends, and an opening that extends entirely through said biodegradable shell proceeding along said length dimension, wherein said opening intersects and proceeds through each of said first and second ends, and wherein an outer perimeter of said opening is defined by said annular inner sidewall; and
calcium chloride retained within said biodegradable shell.
39. An ablation system comprising:
a delivery tube;
a biodegradable shell disposed within said delivery tube; and
calcium chloride retained within said biodegradable shell.
40. An ablation catheter comprising:
a catheter shaft;
a biodegradable shell associated with said catheter shaft; and
calcium chloride retained within said biodegradable shell.
41. The ablation catheter of paragraph 40, wherein said ablation element extends distally from a distal end of said catheter shaft.
42. The ablation catheter of paragraph 40, wherein said ablation element is disposed on an exterior of said catheter shaft.
43. An ablation system comprising:
an expandable balloon;
a first feed lumen extending into an interior of said balloon;
a second feed lumen extending into said interior of said balloon;
an exhaust lumen extending into said interior of said balloon;
a first feed source connected with said first lumen, wherein said first feed source comprises calcium chloride; and
a second feed source connected with said second lumen, wherein said second feed source comprises a liquid.
44. The ablation system of paragraph 43, wherein said first feed source comprises at least one of calcium chloride powder or calcium chloride pallets.
45. The ablation system of any of paragraphs 43-44, wherein said second feed source comprises water.
46. A method of ablating a biological material, comprising:
directing a balloon through a vessel of a patient and to a target location;
expanding said balloon at said target location and into contact with a vessel wall;
directing calcium chloride into said balloon at said target location;
directing a liquid into said balloon at said target location;
reacting said calcium chloride with said liquid within said balloon at said target location;
heating said balloon from said reacting; and
ablating a biological material using said heating.
47. The method of paragraph 46, wherein said calcium chloride comprises at least one of calcium chloride powder or calcium chloride pallets.
48. The method of any of paragraphs 46-47, wherein said liquid is water.
49. The method of any of paragraphs 46-48, wherein said calcium chloride is directed into said balloon through a first lumen and said liquid is directed into said balloon using a separate second lumen.
50. The method of paragraph 49, further comprising directing a flow out of said balloon through a third lumen that is separate from each of said first lumen and said second lumen.
51. The method of any of paragraphs 46-50, directing a flow out of said balloon.
52. The method of any of paragraphs 46-51, wherein said ablating comprises ablating at least one nerve.
53. An ablation system comprising:
an expandable stent disposable within a vessel of a patient, wherein said stent comprises a skeleton and a plurality of openings throughout said skeleton;
a biodegradable shell disposed about at least a portion of an outer perimeter of said stent; and
calcium chloride retained at least within said biodegradable shell.
54. The ablation system of paragraph 53, wherein said biodegradable shell is incorporated only on an exterior of said skeleton.
55. The ablation system of paragraph 53, wherein said biodegradable shell is disposed about an annular portion of said outer perimeter of said stent, wherein said calcium chloride is entirely retained within said biodegradable shell.
56. An ablation system comprising:
an expandable balloon disposable within a vessel of a patient;
a biodegradable shell disposed about an annular outer perimeter of said balloon; and
calcium chloride enclosed within a space between said biodegradable shell and said annular outer sidewall of said balloon.

### BRIEF DESCRIPTION OF THE DRAWINGS

FIG. 1 is a schematic of a human renal anatomy.

FIG. 2A is a perspective view of an ablation element that utilizes a biodegradable shell, wherein at least part of the length of a sidewall of the biodegradable shell is of a reduced wall thickness.

FIG. 2B is a cross-sectional view taken along a length dimension of the ablation element of FIG. 2A.

FIG. 2C is a variation of the ablation element of FIG. 2A, where a sidewall of the biodegradable shell includes one or more annular grooves.

FIG. 2D is a variation of the ablation element of FIG. 2A, where a sidewall of the biodegradable shell includes a groove that spirals along a length of the biodegradable shell.

FIG. 2E is a variation of the ablation element of FIG. 2A, where a sidewall of the biodegradable shell includes a plurality of axially-extending grooves.

FIG. 3A is a perspective view of a hollow ablation element that utilizes a biodegradable shell.

FIG. 3B is a cross-sectional view taken along a length dimension of the ablation element of FIG. 3A.

FIG. 3C is a cross-sectional view taken along a length dimension of a variation of the ablation element of FIG. 3A, where the inner and outer sidewalls are formed from biodegradable materials that degrade at different rates.

FIG. 4A is a perspective view of an ablation element that utilizes a spherical biodegradable shell.

FIG. 4B is a representative extra-vascular positioning of a pair of the ablation elements of FIG. 4A in proximity to a main renal artery.

FIG. 5 is a cross-sectional schematic of a representative guide catheter assembly deployed within a patient's vasculature and that may be used to deliver one or more of the ablation elements disclosed herein to a target location.

FIGS. 6A-6C illustrates a representative delivery device that may be used to deliver one or more of the ablation elements disclosed herein to a target location.

FIG. 7 is a schematic of an extravascularly disposed guide shaft that may be used to deliver one or more of the ablation elements disclosed herein to a target location.

FIG. 8A is a schematic of a catheter that incorporates an ablation element with its catheter shaft, where the ablation element utilizes a biodegradable shell.

FIG. 8B is an enlarged perspective view of the ablation element incorporated by the catheter of FIG. 8A.

FIG. 8C is an enlarged perspective view of a variation of the ablation element of FIGS. 8A and 8B.

FIG. 9A is a schematic of an ablation system that directs multiple feeds into an inflatable balloon.

FIG. 9B is a schematic of the balloon used by the ablation system of FIG. 9A.

FIG. 9C is representative balloon catheter that may utilized by the ablation system of FIG. 9A.

FIG. 10A is a perspective view of an ablation system that includes an expandable stent and at least one biodegradable shell disposed along at least a part of an exterior of the skeleton of the stent.

FIG. 10B is a perspective view of an ablation system that includes an expandable stent and a biodegradable shell disposed about the stent.

FIG. 10C is a schematic of an ablation system that includes an expandable balloon and a biodegradable shell disposed about the balloon.

### DETAILED DESCRIPTION

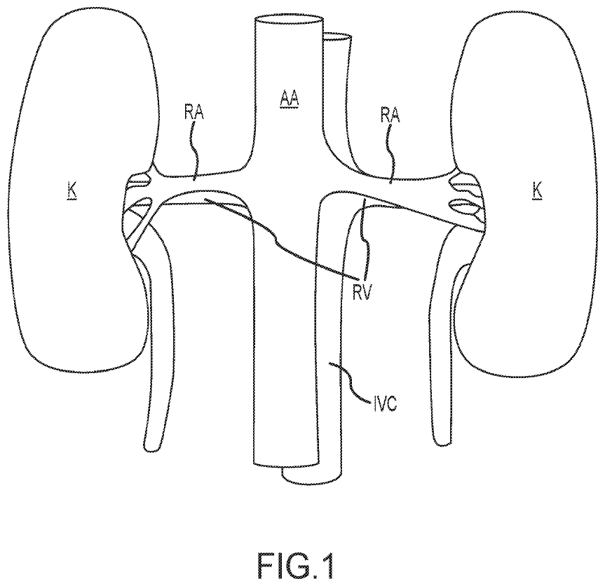

One application for the various ablation elements and/or ablation systems disclosed herein is denervation, including denervating renal nerves. A human renal anatomy is presented in FIG. 1 and includes kidneys K that are supplied with oxygenated blood by renal arteries RA. The kidneys K are connected to the heart by the abdominal aorta AA. Deoxygenated blood flows from the kidneys K to the heart via renal veins RV and the inferior vena cava IVC. Nerves are disposed about the main renal artery, as well as its various branches that extend from the main renal artery to the corresponding kidney K. Additional applications for the various ablation elements and/or ablation systems disclosed herein include tumor ablation, tissue ablation, and the like.

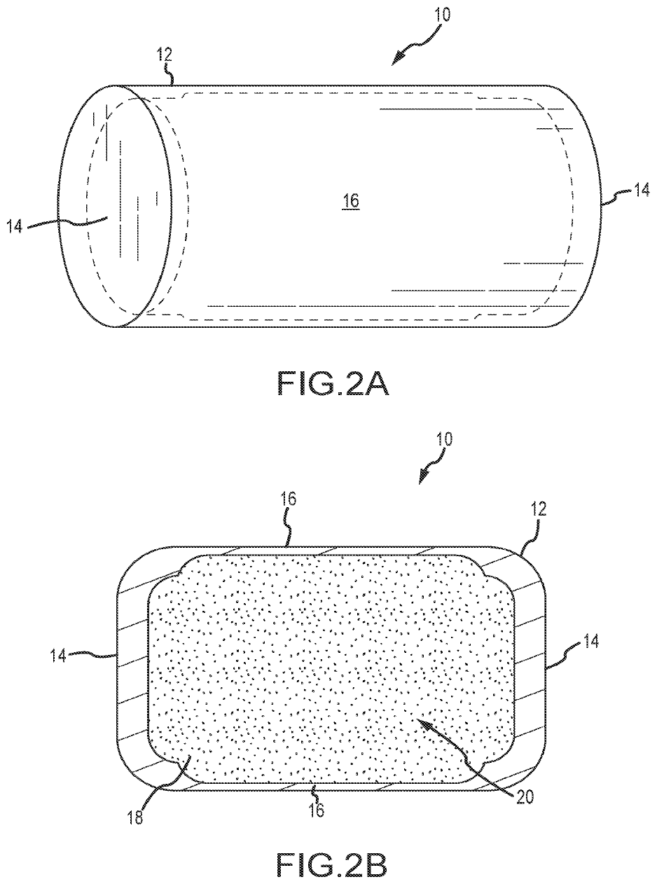

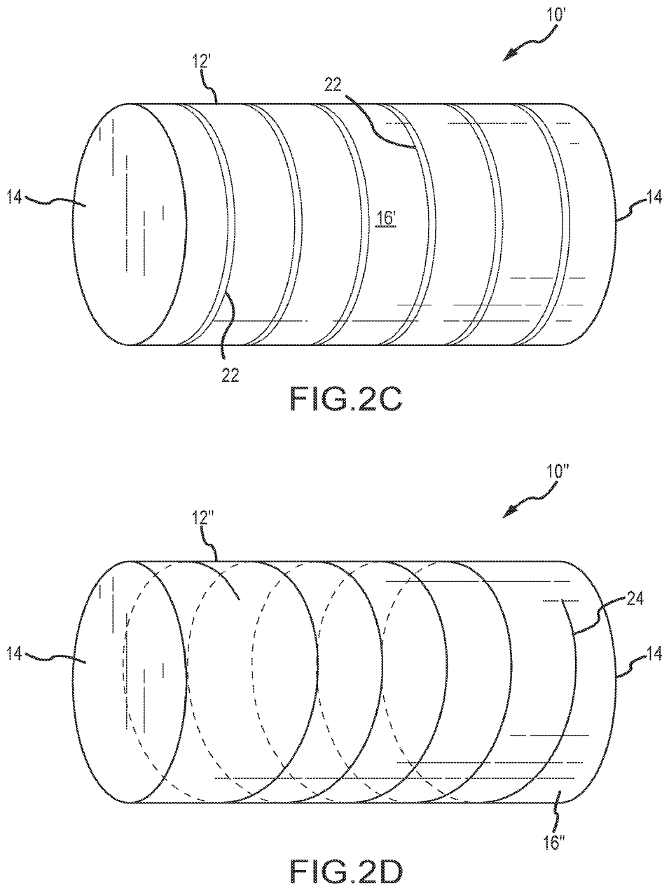

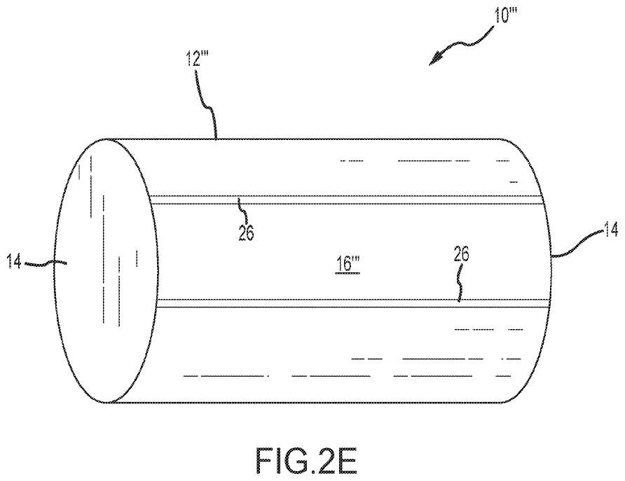

FIGS. 2A-2B disclose an ablation element that is identified by reference numeral 10 and that is configured to be positioned within a vessel of a patient. The ablation element 10 includes a biodegradable shell 12 that may be formed from any appropriate biodegradable material or combination of biodegradable materials (e.g., Gelatin or gelatine). This biodegradable shell 12 includes a pair of ends 14 that are spaced along a length dimension of the biodegradable shell 12. An annular sidewall 16 extends between the two ends 14, with the ends 14 and sidewall 16 collectively defining an enclosed inner storage receptacle 18. Calcium chloride 20 is retained within the inner storage receptacle 18. Representative forms for the calcium chloride 20 include powder, beads, or pallets.

The sidewall 16 of the biodegradable shell 12 may be cylindrical. Another option may be for the outer diameter of the sidewall 16 to be progressively reduced or tapered (e.g., at a constant rate) proceeding from one end 14 of the biodegradable shell 12 to its opposite end 14. This tapering configuration may facilitate engagement of the sidewall 16 with the wall of at least certain vessels (e.g., vessels that become more constricted proceeding along the vasculature).

The wall thickness of at least part of the length of the sidewall 16 of the biodegradable shell 12 is less than a wall thickness of each of the ends 14 of the biodegradable shell 12 in the illustrated embodiment. When the ablation element 10 is positioned within a vessel of the patient, all or at least a portion of the sidewall 16 will be disposed in at least substantially interfacing relation with a wall of this vessel. The ablation element 10 may be retained at a desired target location by a press fit between the ablation element 10 and the wall of the vessel (e.g., the ablation element 10 may be compressible in a direction of a central axis of the ablation element 10′ that corresponds with its length dimension). As at least part of the length of the sidewall 16 has a reduced wall thickness compared to the ends 14, the calcium chloride 20 should be released (by degradation of the sidewall 16) in proximity to the wall of the vessel. A reaction between the released calcium chloride 20 and bodily fluids will generate heat which will ablate the region in proximity to the wall of the vessel (e.g., nerves within this region). Instead of or in combination with above-noted different wall thicknesses, each of the ends 14 may be formed from a different biodegradable material(s) than the annular sidewall 16, where the biodegradable material(s) forming the annular sidewall 16 degrades at a higher/faster rate than the biodegradable material(s) that forms the two ends 14.

A variation of the ablation element 10 of FIG. 2A is illustrated in FIG. 2C and is identified by reference numeral 10′. Corresponding components between the embodiment of FIGS. 2A-2B and FIG. 2C are identified by a common reference numeral, and unless otherwise noted herein the foregoing discussion remains applicable to the ablation element 10′. In the case of the ablation element 10′, the sidewall 16′ of the biodegradable shell 12′ includes at least one groove 22 (e.g., on an exterior surface thereof in the illustrated embodiment). Such a groove 22 provides a reduced wall thickness for the sidewall 16′, which should then in turn degrade prior to a remainder of the biodegradable shell 12′ to release calcium chloride 20 for ablation in accordance with the foregoing (e.g., to provide a corresponding annular ablation). Each groove 22 may be of any appropriate shape/profile. One embodiment has the ablation element 10′ including a plurality of annular grooves 22 (proceeding about the entire perimeter of the sidewall 16′; extending a full 360° about the above-noted central axis of the ablation element 10′ that corresponds with its length dimension), with the annular grooves 22 being spaced along the length dimension of the ablation element 10′.

A variation of the ablation element 10 of FIG. 2A is illustrated in FIG. 2D and is identified by reference numeral 10″. Corresponding components between the embodiment of FIGS. 2A-2B and FIG. 2D are identified by a common reference numeral, and unless otherwise noted herein the foregoing discussion remains applicable to the ablation element 10″. In the case of the ablation element 10″, the sidewall 16″ of the biodegradable shell 12″ includes a groove 24 (e.g., on an exterior surface thereof in the illustrated embodiment). Such a groove 24 provides a reduced wall thickness for the sidewall 16″, which should then in turn degrade prior to a remainder of the biodegradable shell 12″ to release calcium chloride 20 for ablation in accordance with the foregoing. The groove 24 spirals about the annular sidewall 16″ proceeding along the length dimension of the biodegradable shell 10″ to provide a corresponding spiral or helical ablation (e.g., the biodegradable shell 12″ includes at least one spiral or helical groove 24, for instance on its exterior).

A variation of the ablation element 10 of FIGS. 2A-2B is illustrated in FIG. 2E and is identified by reference numeral 10″. Corresponding components between the embodiments of FIGS. 2A-2B and FIG. 2D are identified by a common reference numeral, and unless otherwise noted herein the foregoing discussion remains applicable to the ablation element 10′. In the case of the ablation element 10″, the sidewall 16′″ of the biodegradable shell 12″ includes at least one axially-extending groove 26 (e.g., on an exterior surface thereof in the illustrated embodiment). Such a groove 26 provides a reduced wall thickness for the sidewall 16″, which should then in turn degrade prior to a remainder of the biodegradable shell 12″ to release calcium chloride 20 for ablation in accordance with the foregoing (e.g., to provide a corresponding axially-extending ablation). Each groove 26 may be of any appropriate shape/profile. One embodiment has the ablation element 10″ including a plurality of axially-extending grooves 26 that each proceed along at least a portion of the length of the ablation element 10″, including where the grooves 26 are disposed in at least substantially parallel relation to one another, in at least substantially parallel relation to the above-noted central axis of the ablation element 10′ that corresponds with its length dimension, where any appropriate spacing may be used between adjacent pairs of grooves 26, and/or where the grooves 26 extend along the entire length of the ablation element 10″. In addition to providing for degradation in accordance with the foregoing for release of the calcium chloride 20, the grooves 26 also accommodates passage of bodily fluids (e.g., blood) along the sidewall 16′ to enhance interaction with the released calcium chloride 20.

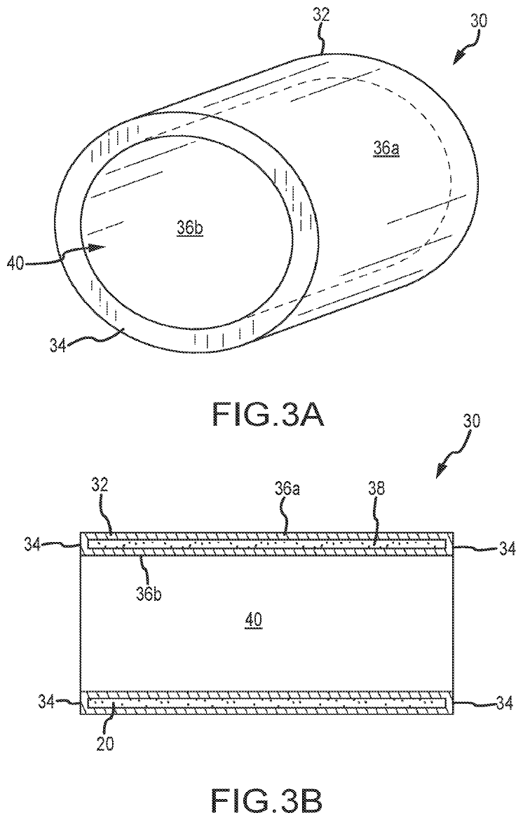

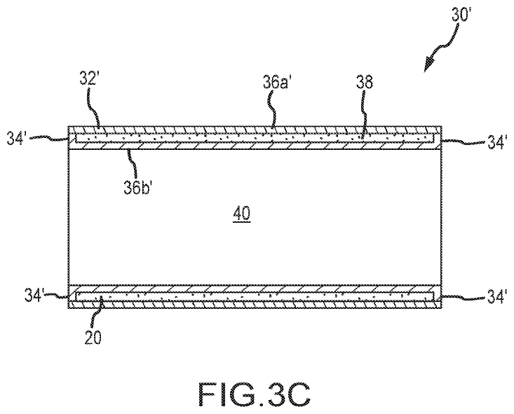

FIGS. 3A-3B disclose an ablation element that is identified by reference numeral 30, that is configured to be positioned within a vessel of a patient, and that incorporates a flow-through feature. The ablation element 30 includes a biodegradable shell 32 that may be formed from any appropriate biodegradable material or combination of biodegradable materials. This biodegradable shell 32 includes a pair of ends 34 that are spaced along a length dimension of the biodegradable shell 32. An annular outer sidewall 36a extends between the two ends 34. An annular inner sidewall 36b of the biodegradable shell 32 is spaced inwardly of the outer sidewall 36a and also extends between the two ends 34. An opening 40 extends completely through the biodegradable shell 32 proceeding along its length dimension, and thereby the opening 40 extends between and intersects with each of the two ends 34 of the ablation element 30 (e.g., the ends 34 may be in the form of annular structures). An outer perimeter of this opening 40 is defined by the inner sidewall 36b. Blood or other bodily fluids may flow through the opening 40.

The opposing ends 34, the outer sidewall 36a, and the inner sidewall 36b collectively define an enclosed inner storage receptacle 38. Calcium chloride 20 in accordance with the foregoing is retained within the inner storage receptacle 38.

The outer sidewall 36a of the biodegradable shell 32 may be cylindrical. Another option may be for the outer diameter of the outer sidewall 36a to be progressively reduced or tapered (e.g., at a constant rate) proceeding from one end 34 of the biodegradable shell 32 to its opposite end 34. This tapering configuration may facilitate engagement of the outer sidewall 36a with the wall of at least certain vessels (e.g., vessels that become more constricted proceeding along the vasculature).

When the ablation element 30 is positioned within a vessel of the patient, all or at least a portion of the outer sidewall 36a will be disposed in at least substantially interfacing relation with a wall of the vessel. The ablation element 30 may be retained at a desired target location by a press fit between the ablation element 30 and the wall of the vessel (e.g., the ablation element 30 may be compressible in a direction of a central axis of the ablation element 30 that corresponds with its length dimension). The wall thickness of at least part of the outer sidewall 36a of the biodegradable shell 12 may be less than a wall thickness of each of the ends 34 of the biodegradable shell 32, the outer sidewall 36a may include one or more grooves 22, and/or the outer sidewall 36a may include a spiral/helical groove 24 in accordance with the foregoing. In any case, the calcium chloride 20 should be released (by degradation of the outer sidewall 36a or at least certain portions thereof (e.g., at each groove 22; along the groove 24) in proximity to the wall of the vessel. A reaction between the released calcium chloride 20 and bodily fluids will generate heat which will ablate the region in proximity to the wall of the vessel (e.g., nerves within this region).

A variation of the ablation element 30 of FIGS. 3A-3B is illustrated in FIG. 3C and is identified by reference numeral 30′. Corresponding components between the embodiments of FIGS. 3A-3B and FIG. 3C are identified by a common reference numeral, and unless otherwise noted herein the foregoing discussion remains applicable to the ablation element 30′. The biodegradable shell 32′ in the case of the ablation element 30′ utilizes multiple biodegradable materials. The annular outer sidewall 36a′ is formed from a biodegradable material (or a combination of biodegradable materials) that degrades at a higher/faster rate than a remainder of the biodegradable shell 32′ (e.g., the ends 34′ and the annular inner sidewall 36b′ of the biodegradable shell 32′ may be formed from one or more biodegradable materials that degrade at a lower/slower rate than the outer sidewall 36a′).

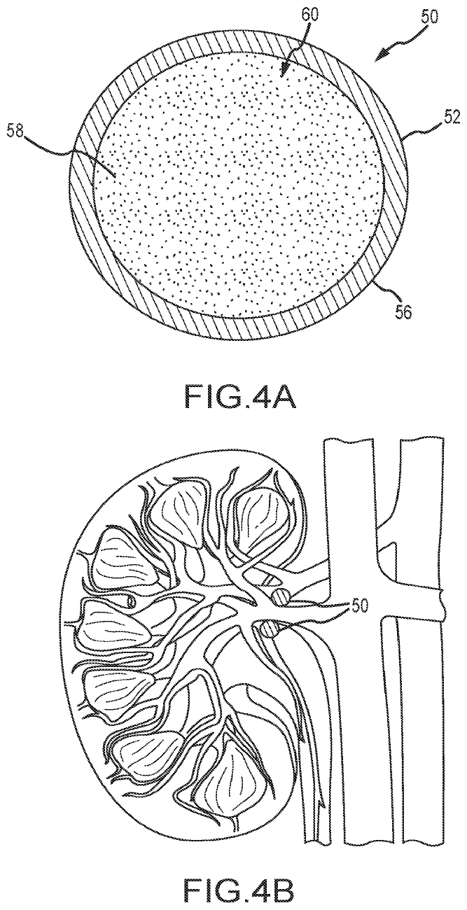

FIG. 4A discloses an ablation element that is identified by reference numeral 50 and that may be configured for extravascular positioning within a patient (e.g., within tissue), although the ablation element 50 could be delivered to a target location within a vessel. The ablation element 50 includes a biodegradable shell 52 that may be formed from any appropriate biodegradable material or combination of biodegradable materials. This biodegradable shell 32 includes an outer wall 56 that is at least generally spherically-shaped (e.g., an outer diameter of no more than about 1 mm). An inner storage receptacle 58 is enclosed by the outer wall 56 and contains calcium chloride 60. The calcium chloride 60 for purposes of the ablation element 50 may be of any appropriate form, including without limitation powder, beads, pallets, or liquid. One or more other agents could be used by the ablation element 50, such as ethanol. FIG. 4B illustrates disposing a number of ablation elements 50 about a main renal artery of a patient. Any appropriate number of ablation elements 50 may be disposed at a particular target location within the body of the patient. When the ablation element 50 is positioned within the body of the patient, the outer wall 56 of the ablation element 50 will degrade to release the calcium chloride 60. A reaction between the released calcium chloride 60 and bodily fluids will generate heat which will ablate the region in proximity to the ablation element 50.

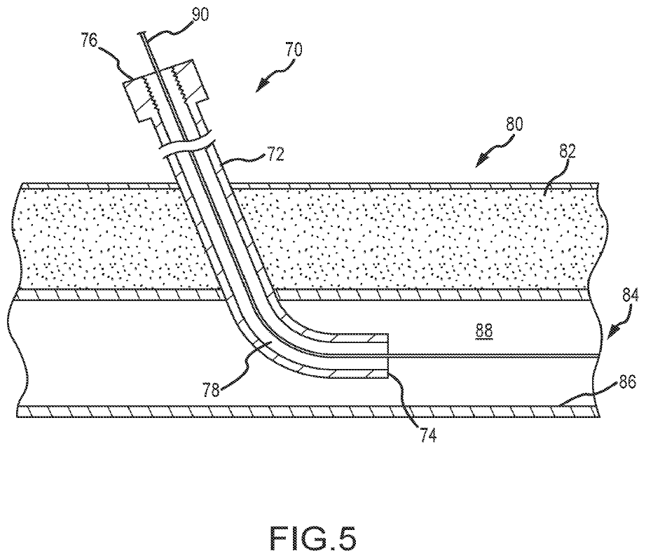

A guide catheter may be used in relation to one or more of the embodiments addressed herein, a representative one of which is illustrated in FIG. 5 and that is identified by reference numeral 70. The guide catheter 70 includes a generally tubular guide shaft 72, which in turn includes a distal end 74, a proximal end 76, and a guide lumen 78 that extends through guide shaft 72 (extending between the distal end 74 and the proximal end 76). The guide catheter 70 is shown as having been directed through tissue 82 of a patient 80, through a wall 86 of a representative vessel 84, and into the lumen 88 of the vessel 84. A guide wire 90 extends through the guide catheter 70 and into the lumen 88 of the vessel 84. As is known in the art: 1) a needle, a short guide wire, and a dilator (removably disposed in the guide lumen 78 of the guide catheter 70 may be used to introduce the guide catheter 70 into the lumen 88 of the vessel 84 (e.g., U.S. Pat. No. 10,271,873); and 2) the guide wire 90 and guide catheter 70 may be advanced along the vessel 84 to the target location, for instance for releasing one or ablation elements at the target location.

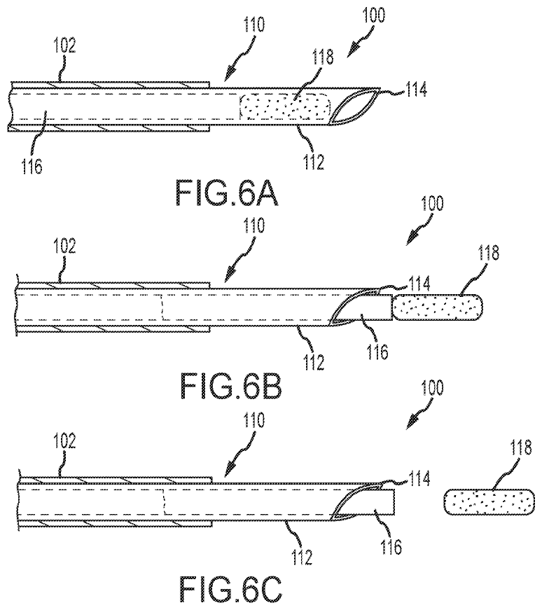

An ablation system is illustrated in FIGS. 6A-6C and is identified by reference numeral 100. In the case where the ablation system 100 is used to deliver one or ablation elements 10/10′/10″ (FIGS. 2A-2D) or to deliver one or ablation elements 30 (FIGS. 3A-3B), the ablation system 100 may a utilize a guide catheter having a guide catheter shaft 102 (e.g., the above-noted guide catheter 70). The ablation system 100 includes a delivery device 110 that is disposed within this guide catheter shaft 102 and includes a delivery shaft 112 and a plunger 116. An ablation element 118 (e.g., ablation element 10, 10′, 10″, 30) may be disposed within the delivery shaft 112 between its distal end 114 and the plunger 116 (e.g., FIG. 6A). The ablation element 118 may be at least somewhat press-fit within the delivery shaft 112. The plunger 116 may be advanced relative to the guide catheter shaft 102 and the delivery shaft 112 to direct the ablation element 118 out of delivery shaft 112 and into the corresponding vessel (FIG. 6B). The delivery shaft 112 and the plunger 116 may then be retracted (FIG. 6C).

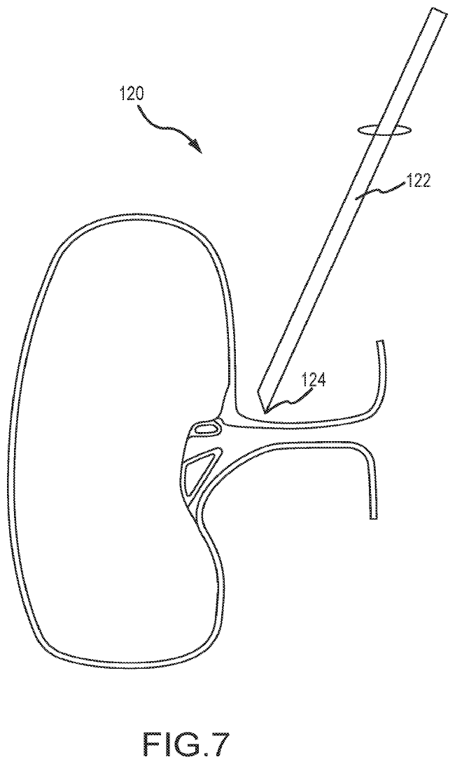

An ablation system is illustrated in FIG. 7 and is identified by reference numeral 120. In the case where the ablation system 120 is used to deliver one or ablation elements 50 (FIG. 4A), the ablation system 120 may utilize the delivery device 110 (FIGS. 6A-6C) and a guide shaft 122 (FIG. 7). The guide shaft 122 may be introduced through the body of the patient (via a completely extravascular approach) to a desired target location (e.g., proximate a renal artery in FIG. 7). The delivery device 110 may be disposed within the guide shaft 122. An ablation element (e.g., ablation element 50) may be disposed within the delivery shaft 112 between its distal end 114 and the plunger 116 (e.g., FIG. 6A; the ablation element 50 may be at least somewhat press-fit within the delivery shaft 112 of the delivery device 110). The plunger 116 may be advanced relative to both the guide shaft 122 and the delivery shaft 112 to direct the ablation element (e.g., ablation element 50) out of delivery shaft 112 and to the target location within the body of the patient (e.g., FIG. 4B and FIG. 7).

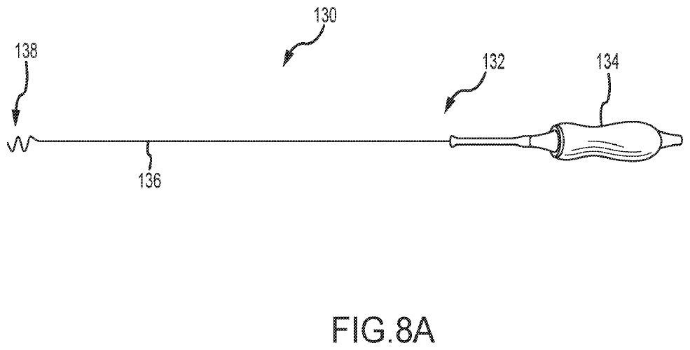

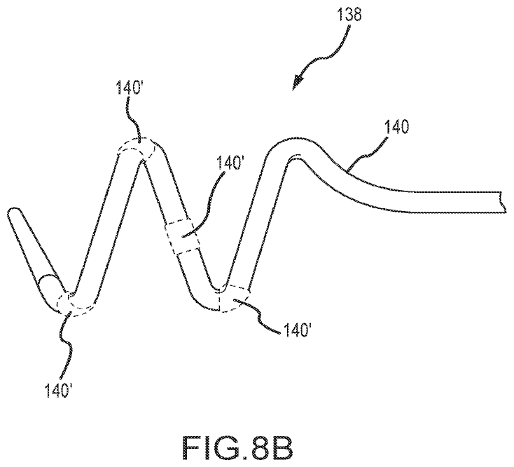

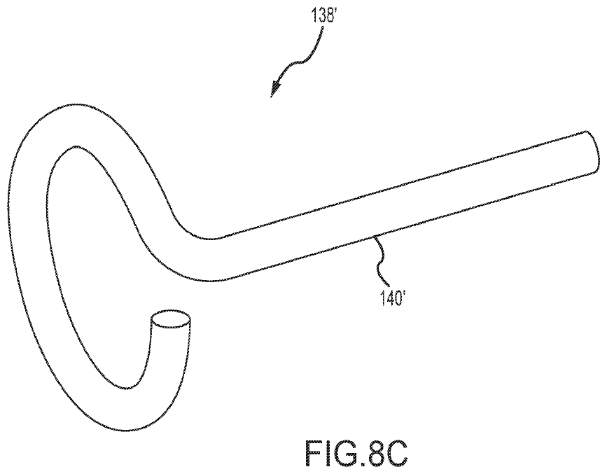

An ablation system is illustrated in FIGS. 8A and 8B and is identified by reference numeral 130. The ablation system 130 is in the form of/utilizes a catheter 132. The catheter 132 includes a catheter handle 134 and a catheter shaft 136 that extends distally from the catheter handle 134. An ablation element 138 is disposed at a distal end of the catheter shaft 136. Alternatively, the ablation element 138 may be characterized as defining a distal end section of the catheter shaft 136. In the illustrated embodiment, the ablation element 138 spirals proceeding along its length dimension. The ablation element 138 includes a biodegradable shell 140, with calcium chloride (e.g., calcium chloride 20) being enclosed within this biodegradable shell 140. That is, the entire distal end section of the catheter 132 may be defined by the biodegradable shell 140. Another option is for a plurality of biodegradable shells 140′ to be disposed on an exterior of the catheter shaft 136 at spaced locations along the catheter shaft 136 (e.g., the distal end section of the catheter shaft 136 may be spirally or helically-shaped, and biodegradable shells 140′ may be spaced along this spiral/helical portion of the catheter shaft 136).

The catheter shaft 136 of the ablation system 130 may be advanced through the vasculature of a patient (e.g., using a guide catheter) and with the ablation element 138 being in a delivery configuration (e.g., compressed to at least a degree from what is shown in FIGS. 8A-8B; a “straighter” profile compared to what is shown in FIGS. 8A-8B). For instance, the ablation element 138 may be disposed within a guide shaft of a guide catheter. In any case when the ablation element 138 is at least generally proximate the target location, the ablation element 138 may be disposed into its deployed configuration of FIGS. 8A-8B, for instance by advancing the catheter shaft 136 relative to a guide shaft of a guide catheter such that the ablation element 138 is now positioned beyond a distal end of this guide shaft. In its deployed configuration, the ablation element 138 should be disposed in at least substantially interfacing relation with a wall of the vessel proceeding along the length dimension of the ablation element (including pressing on the wall of the vessel to retain the ablation element 138 in at least somewhat of a fixed position relative to the wall of the vessel). Degradation of the biodegradable shell 140 of the ablation element 138 will release the calcium chloride 20 in proximity to the wall of the vessel. A reaction between the released calcium chloride 20 and bodily fluids will generate heat which will ablate the region in proximity to the wall of the vessel (e.g., nerves within this region).

A variation of the ablation element 138 of FIGS. 8A/8B is presented in FIG. 8C, and is identified by reference numeral 138′. The ablation element 138′ includes a biodegradable shell 140′ that is in the form of an arcuately extending structure (e.g., at least generally circular, but not extending a full 360° in the illustrated embodiment), versus the spiral configuration of the ablation element 138 of FIGS. 8A-8B. Degradation of the biodegradable shell 140′ will release the calcium chloride 20 in proximity to the wall of the vessel. A reaction between the released calcium chloride 20 and bodily fluids will generate heat which will ablate a single arcuate region in proximity to the wall of the vessel (e.g., nerves within this region).

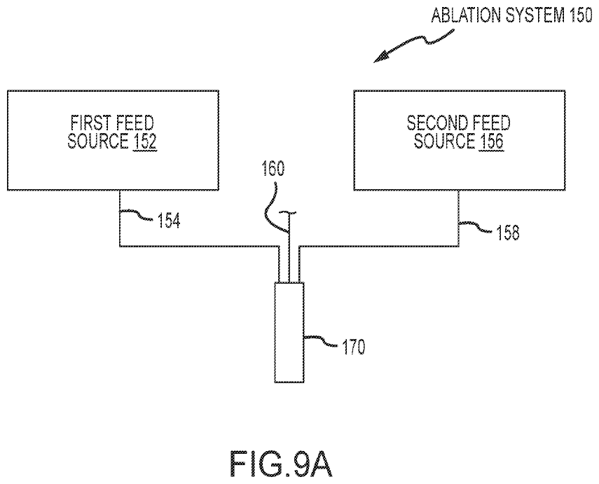

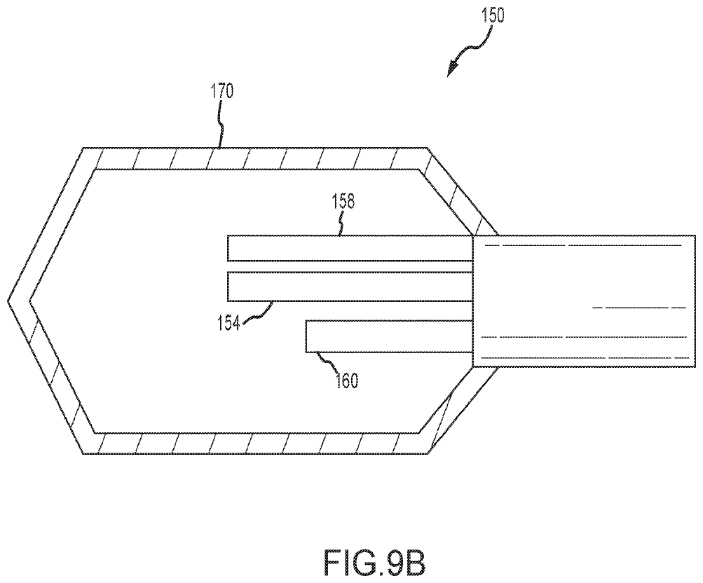

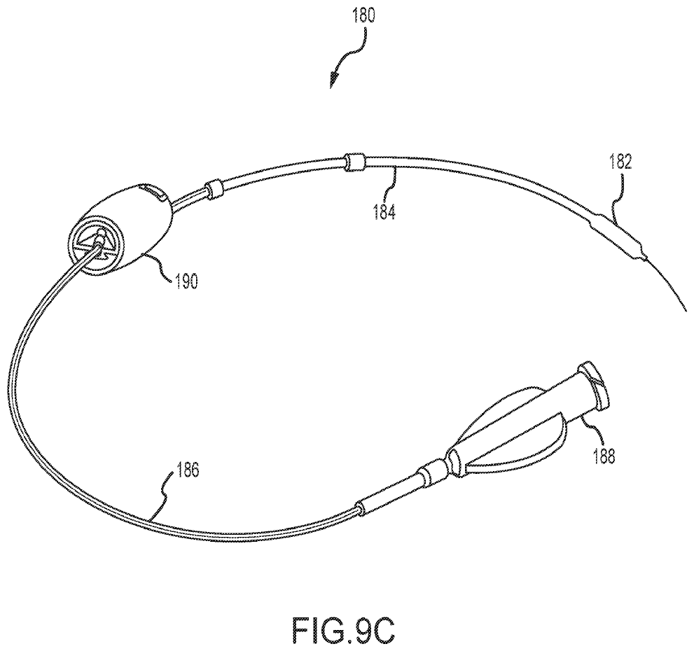

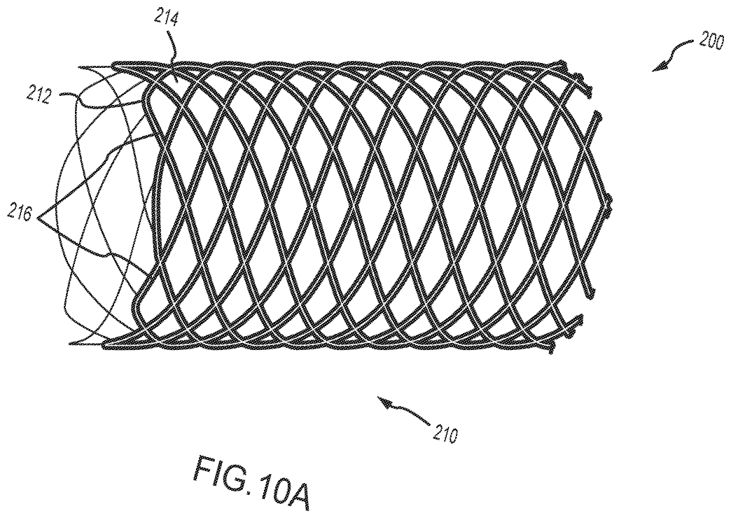

An ablation system is illustrated in FIGS. 9A-9B and is identified by reference numeral 150. Components of the ablation system 150 include a first feed source 152, a first feed line 154 extending from the first feed source 152 to an interior of a balloon 170, a second feed source 156, a second feed line 158 extending from the second feed source 156 to the interior of the balloon 170, and an exhaust line 160 that extends from inside the balloon 170 to a location outside of the balloon 170. Each of the lines 154, 158, and 160 include a lumen to accommodate a corresponding flow. The balloon 170, the first feed line 154, the second feed line 158, and the exhaust line 160 may be incorporated by a balloon catheter (e.g., balloon catheter 180 address below in relation to FIG. 9C).

The first feed source 152 includes a supply of calcium chloride. Representative forms for the calcium chloride of the first feed source 152 include powder or pallets. The second feed source 156 includes a supply of an appropriate liquid, such as water. The balloon 170 will typically be in a contracted state when delivered through the vasculature of a patient to the target location within a vessel. Once the balloon 170 is at the target location within the vessel, the first feed source 152 may be operated to direct a flow of calcium chloride into the balloon 170 and the second feed source 156 may be operated to direct a flow of liquid into the balloon 170. These flows will expand the balloon 170, ultimately into contact with a wall of the vessel. The flows from the first feed source 152 and from the second feed source 156 could be terminated after the balloon 170 has sufficiently engaged the wall of the vessel, or these flows could continue at some appropriate rate (including continuously or intermittently). The ablation system 150 could be configured such that a flow out of the exhaust line 160 is initiated after completion of an ablation, could be configured to accommodate an intermittent flow out of the exhaust line 160, or could be configured to accommodate a continuous flow out of the exhaust line 160.

A reaction between the calcium chloride (first feed source 152) and liquid (second feed source 156) will generate heat within the balloon 170. This heating of the interior of the balloon 170 will heat the wall of the balloon 170 that is in contact with the wall of the vessel. This in turn will ablate a region in proximity to the wall of the vessel being contacted by the balloon 170 (e.g., nerves within this region).

As noted, the balloon 170, the first feed line 154, the second feed line 158, and the exhaust line 160 may be incorporated by a balloon catheter. A representative balloon catheter 180 that could be adapted for use by the ablation system 150 is illustrated in FIG. 9C and is identified by reference numeral 180. The balloon catheter 180 includes a balloon 182 (e.g., balloon 170), a distal shaft 184, a proximal shaft 186, a fitting 188, and a guide member 190. The fitting 188 may be configured to accommodate receipt of separate flows from each of the first feed source 152 and the second feed source 156. A portion of each of the first feed line 154 and the second feed line 158 may extend from the fitting 188, through the proximal shaft 186, through the distal shaft 184, and to the balloon 182.

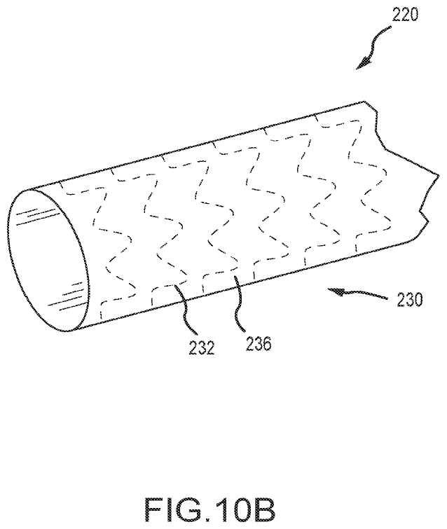

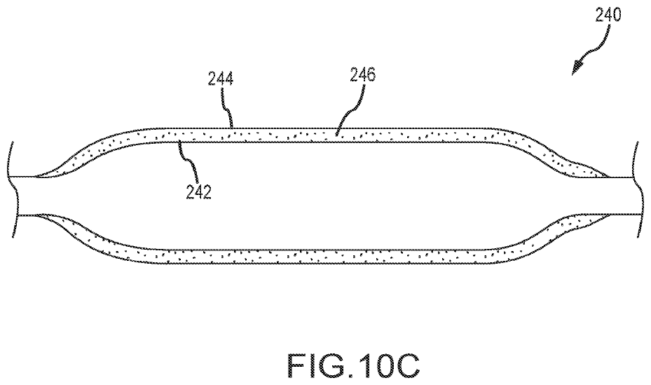

FIG. 10A is a perspective view of an ablation system that is identified by reference numeral 200. The ablation system 200 includes an expandable stent 210 defined by what may be characterized as a skeleton or skeletal structure 212 (e.g., Nitinol). The skeleton 212 may be of any appropriate pattern/configuration, and will typically include various openings 214 that are distributed throughout the skeleton 212 and that may be of any appropriate size and/or configuration. An exterior of at least part of the skeleton 212 includes/incorporates a biodegradable shell 216. The biodegradable shell 216 may be integrated in any appropriate manner with the exterior of the skeleton 212. Calcium chloride is disposed within the biodegradable shell 216. Representative forms for the calcium chloride include powder, beads, or pallets. When the ablation system 200 is delivered to a target location within a vessel of the patient (e.g., using the guide catheter 70 of FIG. 5), the stent 210 may be expanded to dispose the biodegradable shell 216 in contact with the inner wall of the vessel. Degradation of the biodegradable shell 216 will release the calcium chloride in proximity to the wall of the vessel. A reaction between the released calcium chloride and bodily fluids will generate heat which will ablate the region in proximity to the wall of the vessel (e.g., nerves within this region).

FIG. 10B is a perspective view of ablation system that is identified by reference numeral 220. The ablation system 220 includes an expandable stent 230 defined by what may be characterized as a skeleton or skeletal structure 232 (e.g., Nitinol). The skeleton 232 may be of any appropriate pattern/configuration. A biodegradable shell 236 is disposed about the exterior of the stent 230 (and thus “overlying” the various openings in the skeleton 232). The biodegradable shell 236 may be integrated in any appropriate manner with the exterior of the skeleton 232. Calcium chloride is disposed within the biodegradable shell 236. Representative forms for the calcium chloride include powder, beads, or pallets. In any case and when the ablation system 220 is delivered to a target location within a vessel of the patient (e.g., using the guide catheter 70 of FIG. 5), the stent 230 may be expanded to dispose the biodegradable shell 236 in contact with the inner wall of the vessel. Degradation of the biodegradable shell 236 will release the calcium chloride in proximity to the wall of the vessel. A reaction between the released calcium chloride and bodily fluids will generate heat which will ablate the region in proximity to the wall of the vessel (e.g., nerves within this region).

FIG. 10C is a schematic of an ablation system that is identified by reference numeral 240. The ablation system 240 includes an expandable inner balloon 242 and that may be expanded by any appropriate fluid or combination of fluids. A biodegradable outer balloon 244 is disposed about the inner balloon 242. Calcium chloride 246 is disposed between the inner balloon 242 and the outer balloon 244, and is retained within this enclosed space. Representative forms for the calcium chloride include powder, beads, or pallets. When the ablation system 240 is delivered to a target location within a vessel of the patient (e.g., using the guide catheter 70 of FIG. 5; delivering the balloons 242, 244 using an appropriate catheter, such as in accord with the balloon catheter 180 of FIG. 9C), the inner balloon 242 may be expanded to dispose the outer balloon 244 in contact with the inner wall of the vessel. Degradation of the outer balloon 244 will release the calcium chloride in proximity to the wall of the vessel. A reaction between the released calcium chloride and bodily fluids will generate heat which will ablate the region in proximity to the wall of the vessel (e.g., nerves within this region).

Any appropriate biodegradable material or combination of biodegradable materials (e.g., Gelatin or gelatine) may be used to form a biodegradable shell in accordance with the foregoing. One more or appropriate agents/materials may be enclosed within the biodegradable shell to provide the above-noted heating and/or necrosing features upon release from the biodegradable shell, including accounting for the target location of the biodegradable shell within the body. A reaction between the released ablation agent(s) and one or more bodily fluids may generate heat that may be used to ablate at least one or more nerves, to ablate a tumor, or to ablate tissue, in at least certain instances the reaction may be in the form of a chemical reaction that results in necrosis, or both.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

Any feature of any other various aspects addressed in this disclosure that is intended to be limited to a “singular” context or the like will be clearly set forth herein by terms such as “only,” “single,” “limited to,” or the like. Merely introducing a feature in accordance with commonly accepted antecedent basis practice does not limit the corresponding feature to the singular. Moreover, any failure to use phrases such as “at least one” also does not limit the corresponding feature to the singular. Use of the phrase “at least substantially,” “at least generally,” or the like in relation to a particular feature encompasses the corresponding characteristic and insubstantial variations thereof (e.g., indicating that a surface is at least substantially or at least generally flat encompasses the surface actually being flat and insubstantial variations thereof). Finally, a reference of a feature in conjunction with the phrase “in one embodiment” does not limit the use of the feature to a single embodiment.

1. An ablation element for a biological material, the ablation element comprising:
a biodegradable shell comprising first and second end walls and an annular sidewall, wherein the first and second end walls are spaced along a length dimension of the biodegradable shell and with the annular sidewall extending between the first and second end walls; and
calcium chloride retained within the biodegradable shell,
wherein at least a first length section of the annular sidewall has a smaller wall thickness than each of the first and second end walls.

2. The ablation element of claim 1, wherein the annular sidewall extends between the first and second ends, and
wherein the annular sidewall comprises at least one groove defining the smaller wall thickness.

3-7. (canceled)

8. An ablation system comprising:
an expandable balloon;
a first feed lumen extending into an interior of the balloon;
a second feed lumen extending into the interior of the balloon;
an exhaust lumen extending into the interior of the balloon;
a first feed source connected with the first lumen, wherein the first feed source comprises calcium chloride; and
a second feed source connected with the second lumen, wherein the second feed source comprises a liquid.

9. The ablation system of claim 8, wherein the first feed source comprises at least one of calcium chloride powder or calcium chloride pallets.

10. The ablation system of claim 8, wherein the second feed source comprises water.

11-14. (canceled)

15. An ablation system comprising:
an elongate body;
a biodegradable shell disposed within or coupled to the elongate body; and
calcium chloride retained within the biodegradable shell.

16. The ablation system of claim 15, wherein the elongate body comprises a delivery tube, and wherein the biodegradable shell is disposed within the delivery tube.

17. The ablation system of claim 15, wherein the elongate body comprises a catheter shaft, and wherein the biodegradable shell is coupled to the catheter shaft.

18. The ablation system of claim 17, wherein the biodegradable shell extends distally from a distal end of the catheter shaft.

19. The ablation system of claim 17, wherein the biodegradable shell is disposed on an exterior of the catheter shaft.

20. An ablation system comprising:
an expandable member configured to be disposed within a vessel of a patient;
a biodegradable shell disposed about at least a portion of an outer perimeter of the expandable member; and
calcium chloride retained at least within the biodegradable shell.

21. The ablation system of claim 20, wherein the expandable member comprises a stent, the stent comprising skeleton and a plurality of openings throughout the skeleton.

22. The ablation system of claim 21, wherein the biodegradable shell is incorporated only on an exterior of the skeleton.

23. The ablation system of claim 21, wherein the biodegradable shell is disposed about an annular portion of the outer perimeter of the stent, and wherein the calcium chloride is entirely retained within the biodegradable shell.

24. The ablation system of claim 20, wherein the expandable member comprises a balloon, and wherein the calcium chloride is enclosed within a space between the biodegradable shell and an annular outer sidewall of the balloon.
